(19) United States
(12) Patent Application Publication
(10) Pub. No.: US 2013/0224076 A1
(43) Pub. Date: Aug. 29, 2013

(54) MEDICAL DEVICE FOR TREATING A PATIENT

(71) Applicant: DRÄGER MEDICAL GMBH, (US)

(72) Inventors: Hans-Ullrich HANSMANN, Barnitz (DE); Michael RIECKE, Lübeck (DE); Henryk SCHNAARS, Lübeck (DE); Hanno KRETSCHMANN, Hamburg (DE); Ryszard KUMMERFELD, Travemünde (DE)

(73) Assignee: DRÄGER MEDICAL GMBH, Lubeck (DE)

(21) Appl. No.: 13/772,939

(22) Filed: Feb. 21, 2013

(30) Foreign Application Priority Data
Feb. 23, 2012 (DE) 10 2012 003 548.9

(51) Int. Cl. A61L 2/00

(52) U.S. Cl. CPC A61L 2/0094
USPC 422/111; 422/186; 422/119

(57) ABSTRACT

A medical device (10) for treating a patient, with a device body (12), at which at least one functional unit (14) to be operated manually by a treating person is arranged. The device (10) has a disinfection unit (20), which is provided separately from the functional unit and comprises a housing (22) arranged at the device body (12) with a disinfection chamber (26) for receiving at least one hand of the treating person, a plasma generator (28) arranged in the disinfection chamber for generating an atmospheric plasma (P) disinfecting the hand of the treating person and a control unit (50) for actuating the plasma generator (28).

### CROSS REFERENCE TO RELATED APPLICATIONS

This application claims the benefit of priority under 35 U.S.C. §119 of German Patent Application DE 10 2012 003 548.9 filed Feb. 23, 2012, the entire contents of which are incorporated herein by reference.

### FIELD OF THE INVENTION

The present invention pertains to a medical device for treating a patient with a device body, at which at least one functional unit to be operated manually by a treating person is arranged.

### BACKGROUND OF THE INVENTION

Extensive hygienic measures must be taken in medical facilities in order to prevent a direct or indirect transmission of pathogenic microorganisms, hereinafter called simply microorganisms, from patients already contaminated with microorganisms to non-contaminated patients. In particular, microorganism transmission pathways, which become established due to a treating person touching the patient and also a medical device with a functional unit to be operated manually, must be interrupted. There is a risk in this case that another person will become contaminated with microorganisms later by touching the medical device.

Alcohol-containing disinfectants, with which a person treating the patient disinfects his or her hands after contacting the patient, are usually used in practice. Such alcohol-containing disinfectants unfortunately represent a considerable dermatological stress for the hands of this person, so that it is hardly possible, as a rule, to use such a disinfectant after each patient contact.

To avoid excessively frequent use of alcohol-containing disinfectants, the hands of the treating person may also be kept extensively sterile by means of protective gloves. However, it is necessary for this that the treating person discard the protective gloves after each patient contact. It also must be ensured, in particular, that the treating person does not touch any medical device with the protective gloves after a patient contact, because the microorganisms originating from the patient just being treated may be transmitted at first to the medical device and then from the device to other treating persons and finally to other patients. The consistent avoidance of cross contaminations with the use of protective gloves therefore requires the use of a large amount of material and is time-consuming.

Skin disinfection devices, which generate an atmospheric plasma, which is capable of killing microorganisms present on the hands of the treating person, are known from the state of the art. An atmospheric plasma is defined as a plasma whose pressure corresponds to the ambient atmosphere and whose generation does not therefore require pressure chambers provided specifically for that purpose, as they are used to generate a low-pressure plasma. Such a hand disinfection device is described in EP 2 223 704 A1.

The use of a hand disinfection device certainly leads to marked improvements in respect to the necessary hygienic measures. Thus, the plasma exerting disinfecting action on the hands of the treating person causes practically no dermatological stress. The consumption of protective gloves, which can be used only once, can also be drastically reduced by the use of a hand disinfection device.

Nevertheless, a disinfection measure involving the use of such a hand disinfection device does, however, also make it necessary, as a rule, for the treating person, who just touched a patient with his or her hands, to go first to the hand disinfection device and must disinfect his or her hands before he or she may touch a medical device, which is in the immediate vicinity of the patient in space for the treatment of the patient. This makes the treatment process difficult and slows it down.

### SUMMARY OF THE INVENTION

An object of the present invention is to significantly simplify the hygienic measures that must be taken in connection with the treatment of a patient with the use of a medical device of the type mentioned in the introduction to avoid contamination with microorganisms.

This object is accomplished with the present invention in a medical device by a disinfection unit, which is provided separately from a functional unit and comprises a housing arranged on the device body with a disinfection chamber for receiving at least one hand of the treating person, a plasma generator arranged in the disinfection chamber for generating an atmospheric plasma disinfecting the hand of the treating person and a control unit for actuating the plasma generator.

Consequently, the present invention provides for the integration of a disinfection unit operating with an atmospheric plasma in a medical device, which has a functional unit to be operated manually by the treating person for the medical treatment of the patient. After having touched the patient with his or her hands, the treating person can thus disinfect his or her hands before actuating the functional unit arranged at the medical device and thus reliably prevent contamination of the functional unit with microorganisms. The microorganism transmission pathways, which frequently lead via the medical device used to treat the patient, can thus be reliably interrupted. The integration of the disinfection unit operating with an atmospheric plasma in the medical device thus makes it possible to achieve the disinfection of the hands within the treatment process close to the site of handling and in an efficient process. Due to hand disinfection close to the patient directly at the site of treatment, microorganisms are reliably prevented from being transmitted within and outside the treatment room. Since the person is in the vicinity of the medical device during the treatment of the patient anyway, he or she can put the disinfection device into operation at any time without having to move away from the patient. This increases the frequency of application with which disinfection is carried out without causing any essential delay in the treatment of the patient.

In a preferred embodiment, the disinfection unit has a sensor for detecting the hand of the treating person in the disinfection chamber. This sensor preferably generates a detection signal, on the basis of which the control unit actuates the plasma generator.

Provisions are made in an advantageous embodiment for the disinfection unit to have an initialization means for generating an initialization signal and for the control unit to put the plasma generator into operation when receiving the initialization signal and to switch it off after the end of a predetermined operating time. The predetermined operating time is set here such that the atmospheric plasma is generated at a concentration by which the majority of the microorganisms, which have reached the hand of the treating person due to the previous contact with the patient, is killed. A realistic value for the mortality rate brought about by the plasma is, for example, e.g., 1×10⁵, i.e., all of 10⁵ microorganisms are killed except for one. It is obvious that the value indicated above should be considered to be merely an example and higher mortality rates may also be aimed at, e.g., by prolonging the operating time. At any rate, it is the advantage of the above-described embodiment that the control unit switches off the plasma generator automatically after the end of the operating time, without the treating person having to do anything for this himself or herself. This considerably simplifies the disinfection process.

The initialization means is preferably formed by the sensor and the initialization signal is formed by the detection signal. In this embodiment, the sensor recognizes the point in time at which the treating person inserts his or her hand into the disinfection chamber and sends the detection signal to the control unit, which will subsequently put the plasma generator into operation.

However, the initialization means may also be embodied in another manner. For example, the initialization means is formed by two manually actuatable control elements, which are separated from each other in space and are to be actuated simultaneously to generate the initialization signal. Due to the fact that the treating person must actuate two control elements located separated in space from each other simultaneously to put the plasma generator into operation, the plasma generator is prevented with certainty from being put accidentally into operation.

The sensor is preferably formed by a photoelectric cell arranged in the disinfection chamber. Such a photoelectric cell is especially well suited for detecting the hand of the treating person inserted into the disinfection chamber and sending to the control unit a corresponding detection signal, on the basis of which the plasma generator is actuated. However, the sensor arranged in the disinfection chamber is not limited to a photoelectric cell or another sensor of an electro-optical type. Thus, any sensory component can be used basically that is capable of detecting the hand of the treating person in the disinfection chamber.

Provisions are made in a preferred embodiment of the medical device according to the present invention for the disinfection unit to have a light source arranged in the disinfection chamber and for the control unit to put the light source into operation for a predetermined disinfection time, during which the atmospheric plasma exerts disinfecting action. The light source offers the treating person an optical feedback, which indicates, for example, the start and the end of a minimum duration of use. The treating person knows in this case that he or she should insert his or her hands into the disinfection chamber for the duration of the operating time of the light source in order to bring about effective disinfection of the hands. Instead of a light source, which offers an optical feedback, it is also possible to use another component cooperating with the control unit, which brings about a feedback of another type, e.g., an acoustic feedback.

The disinfection unit preferably has an exhaust device for exhausting the atmospheric plasma generated in the disinfection chamber. Pollutants, which are produced by the plasma, can be effectively removed with such an exhaust device, for example, in cooperation with a filter arranged downstream, without entering the ambient air. The operating time of the exhaust device is preferably selected for this such that it immediately follows the duration of use during which the hands of the treating person are disinfected by the atmospheric plasma. It is ensured hereby, on the one hand, that the atmospheric plasma generated by the plasma generator fully exerts its disinfecting action and, on the other hand, that the plasma is rendered harmless as quickly as possible after the disinfecting application.

The control unit preferably generates a log of the data on the operating state of the disinfection unit. Such data contain, for example, information on the duration of use, during which the hands of the treating person are exposed to the plasma, characteristics that characterize the plasma generated by the plasma generator, e.g., the field intensity and/or voltage with which the plasma is generated, duration of plasma generation, date and time of the plasma treatment, etc.

The medical device preferably has an interface for outputting logged data contained in the log. The data can be outputted via this interface to an output device, e.g., a monitor, which is preferably part of the medical device itself.

The housing of the disinfection unit is preferably a cuboid box, which has an opening to the disinfection chamber. The housing may be designed, for example, as a drawer module.

The medical device according to the present invention is, for example, an anesthesia device, a respirator, a patient monitor, a carrying system for receiving medical devices, a syringe pump or an infusion pump. The functional unit, which is to be operated manually at this device by the treating person, may be any device part, e.g., a touch-sensitive screen, which the person touches to change the display.

The present invention will be explained in more detail below on the basis of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

FIG. 1 is a schematic view showing a medical device according to the present invention as an exemplary embodiment; and

FIG. 2 is a flow chart, which illustrates an example of the mode of operation of the device shown in FIG. 1.

### DESCRIPTION OF THE PREFERRED EMBODIMENTS

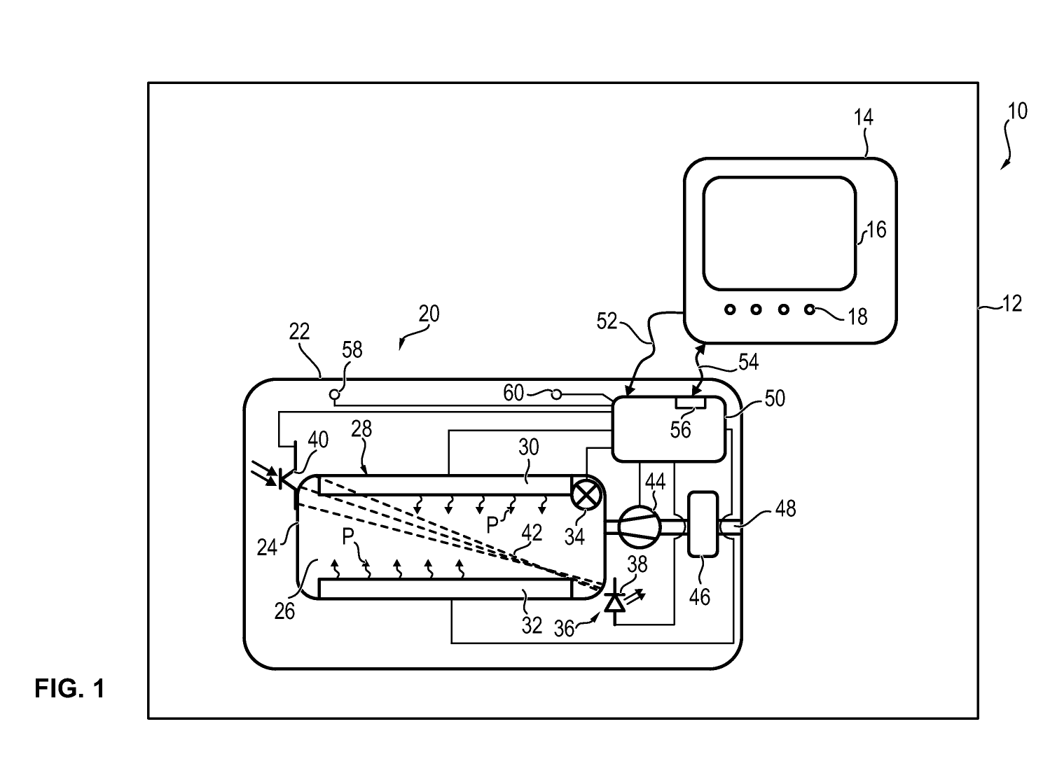

Referring to the drawings in particular, in a schematic view, FIG. 1 shows a medical device 10 as an exemplary embodiment. The medical device 10 is, for example, a respirator (also known as a ventilator), which is used in intensive care to respirate (ventilate) patients with insufficient spontaneous breathing. The medical device 10 has a device body 12, which is shown purely schematically in FIG. 1 and on which a plurality of functional units are arranged, which are actuated manually by a person for treating the patient. Only a monitor 14, which has a touch-sensitive screen 16 as well as a plurality of control buttons 18, is shown purely as an example for these functional units in FIG. 1.

Furthermore, a disinfection unit 20, which the treating person can use to disinfect his or her hands after treating the patient in the manner to be described later, is arranged at the device body 12. Disinfection unit 20 has a housing 22, which is a cuboid box in this exemplary embodiment, which has an essentially rectangular opening 24 on its front side. Housing 22 is designed as a drawer module, which is guided movably on the device body 12. The interior space of housing 22, which is accessible through the opening 24, forms a disinfection chamber 26, in which a plasma generator 28 is accommodated. The opening 24 and disinfection chamber 26 are dimensioned such that the treating person can easily insert both hands into the disinfection chamber 26 through the opening 24. In this exemplary embodiment, housing 22 has a width of about 20 cm, a height of about 5 cm and a depth of about 20 cm. It is apparent that these data should be considered to be an example.

Plasma generator 28 contains two electrode arrays 30 and 32, which face each other and one of which is arranged on the upper side and the other on the underside of the disinfection chamber 26. The electrode arrays 30 and 32 are each designed to generate an atmospheric plasma P having disinfecting action in the known manner with the application of an alternating electric voltage. The design and mode of operation of the plasma generator 28 used in the device 10 according to the present invention correspond essentially to those of the generator known from EP 2 223 704 A1.

Furthermore, a light source 34 and a photoelectric cell 36, which is formed from a light-emitting diode 38 and a phototransistor 40, are located in the disinfection chamber 26. The photoelectric cell 36 recognizes the interruption of a light beam 42 sent by the light-emitting diode 38 to the photodiode 40.

Plasma generator 28 is capable of producing the atmospheric plasma P from air. Mainly the oxygen of the air is ionized in plasma generation, so that ozone that can be for the disinfecting plasma treatment is formed. The ozone is stable for about 30 sec after its generation in this exemplary embodiment. If has disinfection action during this period.

The disinfection unit 20 has, furthermore, a blower 44, which is in connection with the disinfection chamber 26 and is used to draw off the plasma P after the plasma treatment from the disinfection chamber 26 to feed it to a carbon filter 46. The ozone contained in the plasma P is again converted into harmless atmospheric oxygen in the carbon filter 46 and this oxygen is subsequently released into the ambient air via a gas outlet 48.

The disinfection unit 20 has a control unit 50, which is electrically connected to both electrode arrays 30, 32, light source 34, light-emitting diode 36, phototransistor 40 and blower 44 for actuation. Control unit 50 is coupled, furthermore, with the monitor 14 via a supply line 52 and a data line 54. The control unit 50 is supplied with electricity via the supply line 52. The control unit 50 is thus able to supply, in turn, the components of the disinfection unit 20 that are coupled with it with electricity. The control unit 50 is connected, furthermore, to the monitor 14 via a bidirectional data line. Finally, two start buttons 58 and 60, which can be lit and are likewise connected to the control unit 50, are located on the front side of the housing 22, which said front side is accessible to the treating person.

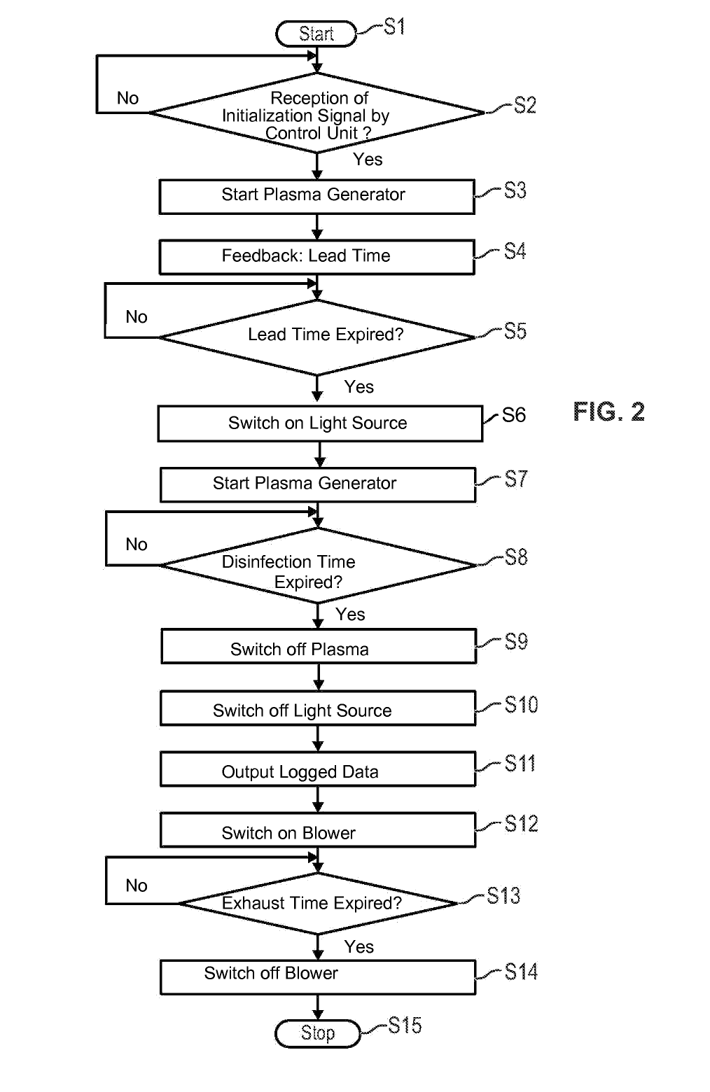

How the medical device 10 can be used to disinfect the hands during the treatment of a patient will be explained as an example below with reference to the flow chart shown in FIG. 2.

The process shown in FIG. 2 starts in step S1. It is assumed for the exemplary embodiment shown in FIG. 1 that the disinfection unit 20 is also ready to operate when the monitor 14 is put into operation, because the disinfection unit is supplied with electricity from the monitor 14 via the supply line 52. In step S2, the control unit 50 checks whether it has received an initialization signal, which indicates that hand disinfection shall be performed. This initialization signal is generated in the exemplary embodiment according to FIG. 1 by the treating person actuating the two start buttons 58 and 60 simultaneously. If such a simultaneous actuation of the start buttons 58 and 60 is detected by receiving the initialization signal, control unit 50 starts the plasma generator 28 in step S3 by putting the two electrode arrays 30 and 32 into operation. The treating person then inserts his or her hands, which are to be disinfected, into the disinfection chamber 26 through the opening 24.

The putting into operation of the plasma generator 28 is reported to the treating person by means of a corresponding feedback in step S4. This happens in this exemplary embodiment by the two start buttons 58 and 60, which can be lit, are caused by the control unit 50 to blink. The blinking of the two start buttons 58 and 60 signals that a lead time, during which the electrode arrays 30 and 32 successively increase the quantity of active plasma P in the disinfection chamber 26 to the extent that a quantity of plasma sufficient for the disinfection of the hands is available in the disinfection chamber 26, has passed. A certain lead time, which is known to be sufficient for generating a sufficient quantity of plasma, e.g., 10 sec, is preset in this exemplary embodiment.

It is checked in step S5 whether the preset lead time has already expired. If it has, the control unit 50 switches on in step S6 the light source 34 arranged in the disinfection chamber, as a result of which the disinfection chamber 26 is lit. This lighting of the disinfection chamber 26 signals to the treating person that the plasma P generated in the disinfection chamber 26 now has full disinfecting action.

With the switching on of light source 34, the control unit 50 detects in step S7 a detection signal, which is generated by the photoelectric cell 36 and signals whether the light beam 42 sent to the light-emitting diode 38 to the phototransistor 40 is interrupted or not. If the light beam 42 is interrupted, it is inferred from this that the hands of the treating person are present in the disinfection chamber 26.

It is checked in step S8 whether a preset disinfection time has passed or not. This disinfection time is preset such that sufficient disinfection of the hands can be expected if the atmospheric plasma P generated in the disinfection chamber 28 acts on the hands during the entire disinfection time. A realistic value for this disinfection time, which should be considered to be only an example, is about 30 sec.

The loop formed from steps S7 and S8 makes a log during the entire disinfection time to show whether the hands of the treating person are indeed in the disinfection chamber 26 or not.

If it is determined in step S8 that the disinfection time has passed, the control unit 50 switches off the plasma generator 28 in step S9. The light source 34 is also put out of operation in step S10 immediately after the plasma generator 28 is switched off, as a result of which it is signaled to the treating person that the preset disinfection time has passed and the disinfection process has ended. The treating person can thus again remove his or her hands from the disinfection chamber 26.

In step S11, the control device 50 provides and output of the logged data obtained from the monitoring of the detection signal via its data interface 56 via data line 54 to monitor 14, on the screen 16 of which the logged data are displayed. These logged data contain, for example, further information, e.g., data on the plasma produced, date and time of disinfection, etc., besides a hand exposure time, which indicates how long the treating person held his or her hands in the plasma P having disinfecting action.

In step S12, which immediately follows in time the switching off of the plasma generator 28, the control unit 50 switches on the blower 38. As a result, blower 37 draws the plasma P, which contains ozone, as was mentioned before, from the disinfection chamber 26 and sends it to the carbon filter 46, in which the ozone is converted into harmless oxygen. The gas passing through the carbon filter 46 is finally released into the ambient air via the gas outlet 48.

It is checked in step S13 whether a preset exhaust time, during which blower 44 is in operation, has expired or not. This exhaust time is preset such that more or less complete conversion of the ozone generated in the disinfection chamber 26 into oxygen by the carbon filter 46 is ensured.

If it is determined in step S13 that the exhaust time has expired, control unit 50 switches off the blower 44 in step S14, after which the disinfection process ends in step S15.

It is obvious that the disinfection process explained above with reference to FIG. 2 should be considered to be merely an example. Many different modifications of this process are conceivable. For example, the initialization signal is generated in the example according to FIG. 2 by the two start buttons 58 and 60 arranged separated from each other in space being pressed simultaneously by the treating person. However, it is also conceivable to let the initialization signal be generated by the photoelectric cell 36. The photoelectric cell 36 is thus able to recognize the insertion of the hands of the treating person into the disinfection chamber 26 on the basis of an interruption of the light beam 42. The detection signal sent by the photoelectric cell 36 to the control unit 50 can thus be used at the same time as an initialization signal, which signals that the treating person has started the disinfection process by having inserted his or her hands into the disinfection chamber 26. The start buttons 58 and 60 may be eliminated in such a variant.

The feedback, which informs the treating person of the putting into operation of the plasma generator 28, is generated optically in the example shown in FIG. 2 by the two start buttons 58 and 60 being caused to blink. It is, of course, also possible to provide another type of feedback, e.g., an acoustic feedback with the use of a suitable sound generator or a feedback displayed on the screen 16 of monitor 14.

In the example shown in FIG. 2, the plasma generator 28 is started in step S3 and is then also kept in operation after the end of the lead time, i.e., during the disinfection time. However, it is also possible to switch off the plasma generator 28 already after the end of the lead time if it is ensured that the plasma generated during the lead time will be sufficient during the disinfection time to achieve the desired disinfecting action.

In the exemplary embodiment shown, the disinfection unit 20 is supplied with electricity via the supply line 52 from monitor 14. However, it is also conceivable to equip the disinfecting unit 20 with an energy source of its own. This is indicated, for example, if the medical device 10 has, besides the disinfecting unit 20, only functional units that have no energy supply themselves, e.g., if the medical device 10 is a purely mechanical device, e.g., a carrying system for receiving other medical devices.

While specific embodiments of the invention have been shown and described in detail to illustrate the application of the principles of the invention, it will be understood that the invention may be embodied otherwise without departing from such principles.

### APPENDIX

LIST OF REFERENCE NUMBERS

| | |
|---|---|
| 10 | Medical device |
| 12 | Device body |
| 14 | Monitor |
| 16 | Touch-sensitive |
| | screen |
| 18 | Control buttons |
| 20 | Disinfection unit |
| 22 | Housing |
| 24 | Opening |
| 26 | Disinfection chamber |
| 28 | Plasma generator |
| 30, 32 | Electrode arrays |
| 34 | Lamp |
| 36 | Photoelectric cell |
| 38 | Light-emitting diode |
| 40 | Phototransistor |
| 42 | Light beam |
| 44 | Blower |
| 46 | Filter |
| 48 | Gas outlet |
| 50 | Control unit |
| 52 | Supply line |
| 54 | Data line |
| 56 | Interface |
| 58, 60 | Start buttons |
| S1 through S15 | Steps of the |
| | disinfection process |

What is claimed is:

1. A medical device for treating a patient, the medical device comprising:
a device body;
a functional unit to be operated by a treating person manually, said functional unit being arranged at said device body; and
a disinfection unit provided separately from said functional unit, said disinfection unit comprising a housing arranged at said device body, a disinfection chamber for receiving at least one hand of the treating person, a plasma generator arranged in said disinfection chamber for generating an atmospheric plasma disinfecting the hand of the treating person and a control unit for actuating said plasma generator.

2. A medical device in accordance with claim 1, wherein said disinfection unit has a sensor for detecting the hand of the treating person in said disinfection chamber.

3. A medical device in accordance with claim 2, wherein said control unit actuates said plasma generator as a function of a detection signal generated by said sensor.

4. A medical device in accordance with claim 1, wherein said disinfection unit has an initializing means for generating an initialization signal and said control unit puts said plasma generator into operation upon receiving said initialization signal and switches it off after an end of a predetermined operating time.

5. A medical device in accordance with claim 4, wherein:
said initialization means is formed by a sensor for detecting the hand of the treating person in the disinfection chamber;
said control unit actuates said plasma generator as a function of a detection signal generated by said sensor; and
said initialization signal is formed by said detection signal.

6. A medical device in accordance with claim 4, wherein said initialization means is formed by two manually actuatable control elements which are separated from each other in space and are to be actuated simultaneously to generate said initialization signal.

7. A medical device in accordance with claim 2, wherein said sensor is formed by a photoelectric cell arranged in said disinfection chamber.

8. A medical device in accordance with claim 1, wherein:
said disinfection unit has a light source arranged in said disinfection chamber; and
said control unit puts said light source into operation for a predetermined disinfection time, during which the atmospheric plasma exerts disinfecting action.

9. A medical device in accordance with claim 1, wherein said disinfection unit has an exhaust device for exhausting the atmospheric plasma generated in said disinfection chamber.

10. A medical device in accordance with claim 9, wherein said disinfection unit has a filter, which is arranged between said exhaust device and a gas outlet.

11. A medical device in accordance with claim 1, wherein said control unit makes a log of data on the operating state of said disinfection unit.

12. A medical device in accordance with claim 11, wherein said disinfection unit has an interface for outputting the logged data.

13. A medical device in accordance with claim 1, wherein said housing is a cuboid box, which has an opening to said disinfection chamber.

14. A medical device in accordance with claim 1, wherein said housing is designed as a drawer module.

15. A medical device in accordance with claim 1, wherein the medical device is part of one or more of an anesthesia device, a respirator, a patient monitor, a carrying system for receiving medical devices, a syringe pump and an infusion pump.

16. A medical device for treating a patient, the medical device comprising:
a device body;
a functional unit to be operated by a treating person manually, said functional unit being operatively connected to said device body; and
a disinfection unit provided separately from said functional unit, said disinfection unit comprising a housing operatively connected to said device body, a disinfection chamber for, a plasma generator generating an atmospheric plasma in said disinfection chamber for disinfecting some part of the treating person and a control unit for actuating said plasma generator.

17. A medical device in accordance with claim 16, wherein:
said disinfection unit has a sensor for detecting the part of the treating person in said disinfection chamber; and
said control unit actuates said plasma generator as a function of a detection signal generated by said sensor.

18. A medical device in accordance with claim 16, wherein:
said disinfection unit has an initializing device generating an initialization signal;
said initialization device is formed by at least one of a sensor for detecting the part of the treating person in the disinfection chamber, to generate said initialization signal, and two manually actuatable control elements which are separated from each other in space, and are to be actuated simultaneously to generate said initialization signal; and
said control unit actuates said plasma generator as a function of said initialization signal.

19. A medical device in accordance with claim 16, wherein:
said disinfection unit has a light source arranged in said disinfection chamber; and
said control unit puts said light source into operation for a predetermined disinfection time, during which the atmospheric plasma exerts disinfecting action.

20. A medical device in accordance with claim 16, wherein said disinfection unit has an exhaust device for exhausting the atmospheric plasma generated in said disinfection chamber.
